(19) United States
(12) Patent Application Publication
(10) Pub. No.: US 2009/0270918 A1
(43) Pub. Date: Oct. 29, 2009

(54) Surgical Intervertebral Implant Forming a Swivel Joint

(76) Inventors: David Attia, Montelimar (FR); Thomas Droulout, Chatou (FR); Dominique Petit, Verton (FR)

Correspondence Address: IP GROUP OF DLA PIPER LLP (US), ONE LIBERTY PLACE, 1650 MARKET ST, SUITE 4900, PHILADELPHIA, PA, 19103, US

(21) Appl. No.: 11/887,970

(22) PCT Filed: Apr. 10, 2006
(86) PCT No.: PCT/FR2006/000791

§ 371 Date: Nov. 26, 2008

(30) Foreign Application Priority Data
Apr. 8, 2005 (FR) 05/03524

(51) Int. Cl. A61B 17/70; A61F 2/44

(52) U.S. Cl. 606/248; 623/17.11

(57) ABSTRACT

A surgical implant that supports and assists movement adjacent vertebrae including a top part and a bottom part associated with the spinous process of one of the vertebrae, each part including means for association with one process and reciprocal means or rotational association with the other part and forming a swivel joint.

### RELATED APPLICATION

This is a §371 of International Application No. PCT/FR2006/000791, with an international filing date of Apr. 10, 2006 (WO 2006/106246 A2, published Oct. 12, 2006), which is based on French Patent Application No. 05/03524, filed Apr. 8, 2005.

### TECHNICAL FIELD

This disclosure relates to a surgical implant intended to support and assist the movement of two vertebrae that are adjacent to one another.

### BACKGROUND

Ageing and the wear of vertebral discs, whether spontaneous or following herniated disc operations, results in sagging of the spaces separating the vertebrae, which produces excessive pressure on the posterior articular cartilage and constriction of the so-called “intervertebral foramina” which the nerve roots pass through. This leads to pain in the lumbar area or in the lower membranes (sciatica, for example).

EP 0392124 describes a surgical implant intended to prevent mutual contact between vertebrae comprising an intervertebral wedge. The wedge comprises top and bottom grooves intended to receive the spinous processes of the vertebrae located on either side of the wedge. Such a wedge prevents contact between the vertebrae while allowing the relative movement of the latter.

However, that surgical implant has several disadvantages. Indeed, the relative movement of the vertebrae is not assisted by the implant and the vertebrae can move in relation to one another by ensuring free play for the support of the spinous processes in the grooves. However, such free play does not guarantee efficient support of the wedge between the vertebrae and the spinous processes may become incorrectly positioned in the grooves, thus reducing the efficiency of the implant. In addition, such free play can also cause movements that are harmful for the correct operation of the vertebral column.

### SUMMARY

We provide a surgical implant that supports and assists movement of adjacent vertebrae including a top part and a bottom part associated with the spinous process of one of the vertebrae, each part including means for association with one process and reciprocal means for rotational association with the other part and forming a swivel joint.

We also provide a linking system between at least an imlant and/or a bone structure including a flexible band, at least one attachment element for the band that attaches the band around all or part of the parts, the attachment element including a hole arranged so that the band passes through it, and tightening means that adjust tightness of the band around the parts.

### BRIEF DESCRIPTION OF THE DRAWINGS

Other special features and advantages of the implants will become apparent from the following description, made in reference to the following figures, in which:

FIG. 1 is a diagrammatic perspective representation of a surgical implant;

FIG. 2 is a diagrammatic front representation of the implant of FIG. 1;

FIG. 3 is a diagrammatic partial cross-section representation of the implant along the III-III axis of FIG. 1;

FIG. 4 is a diagrammatic partial cross-section representation of the implant along the IV-IV axis of FIG. 2;

FIG. 5 is a diagrammatic perspective representation of two consecutive implants associated with one another;

FIG. 6 is a diagrammatic perspective representation of two consecutive implants solidly attached by a part forming a rod;

FIG. 7 is a diagrammatic perspective representation of two consecutive implants solidly attached by an intermediate part;

FIG. 8 is a diagrammatic perspective representation of an association system according to a first configuration;

FIG. 9 is a diagrammatic cross-section representation of the association system along the IX-IX axis of FIG. 8;

FIG. 10 is a diagrammatic cross-section representation of the association system of FIG. 9, in an attachment position;

FIG. 11 is a diagrammatic perspective representation of an association system according to a third configuration;

FIG. 12 is a diagrammatic cross-section representation of the association system along the XII-XII axis of FIG. 11;

FIG. 13 is a diagrammatic perspective representation of an association system according to a fourth configuration; and

FIG. 14 is a diagrammatic cross-section representation of the association system along the XIV-XIV axis of FIG. 13.

### DETAILED DESCRIPTION

We provide a surgical implant that supports and assists the movement of two vertebrae that are consecutive/adjacent to one another, assisting the relative movement between the vertebrae and thus allowing the implant to tighten effectively around the spinous processes of vertebrae.

The implant comprises a top part and a bottom part intended to each be associated with the spinous process of one of the vertebrae, the parts each comprising means for association with one process and reciprocal means for rotational association with the other part, the means forming a swivel joint.

The surgical implant also comprises a viscoelastic element interposed between the top and bottom parts and arranged to absorb the energy from impacts and to allow relative movement between the vertebrae. This interposed intermediate element, which has a certain elasticity, enables deformation upon compression and distraction between the top and bottom parts. Thus, the viscoelastic-element allows flexion, extension or even lateral inflexion of the vertebral column.

The swivel joint formed by the reciprocal association means makes it possible to form an articulation assisting the movement of the vertebrae between one another, the vertebral column thus recovering its natural mobility with no excessive movements, mobility identical to that of a healthy spine. It is therefore possible to do without the clearance between the means for association with the spinous processes and these processes to allow the relative movement of these vertebrae, which guarantees better support for the implant between the vertebrae.

The means for association with the processes have an anatomical shape. In fact, they are adapted to the shape of the spinous processes with which they are intended to be associated. This provides better stability of the means for association with the processes and the operation required to modify the shape of the spinous processes is less serious.

According to another aspect, we provide a linking system intended to associate at least two parts to one another, the parts being either an implant or a bone structure. For this purpose, the linking system comprises a flexible band, at least one band attachment element for attaching the band around all or part of the parts, the attachment element comprising a hole through which the band can pass, and tightening means for adjusting the tightness of the band around the parts.

Advantageously, the notches of the band and the projection of the attachment element are arranged to allow the band to slide in one direction in the attachment element when it passes through the hole.

According to one particular configuration, the attachment element is arranged on one end of the band. Advantageously, the projection formed in the hole extends in the direction of the surface of the band opposite the notched surface.

According to another configuration, the linking system comprises two attachment elements which one of the ends of the band can respectively pass through, each attachment element being configured to slide in one direction on the band, in the direction opposite the end on which it is mounted. Advantageously, the band comprises two distinct notched sections on one of its surfaces, each notched section being intended to cooperate with one of the attachment elements.

According to another configuration, the attachment elements are connected and arranged with one another to form means for association with one of the parts. Advantageously, they have a shape that substantially complements that of the section of the part with which they are intended to be associated, in particular to provide better stability of the association means on the part with which they are in contact.

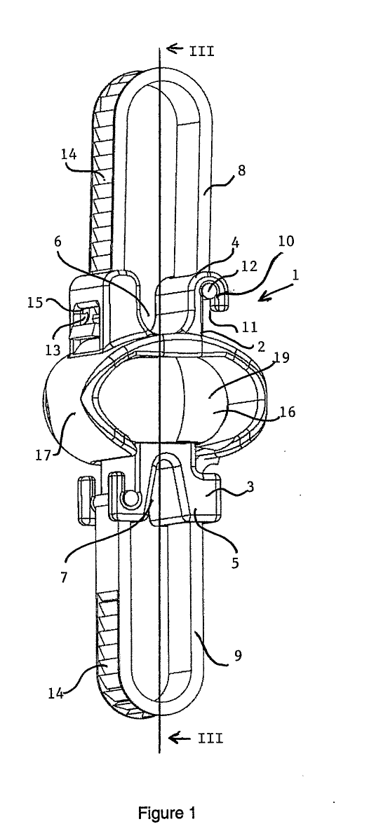

In reference to FIGS. 1 to 4, we describe a surgical implant 1 comprising a top part 2 and a bottom part 3 intended to each be associated with the spinous process of two consecutive vertebrae positioned one above the other.

The top 2 and bottom 3 parts are preferably made from a plastic material such as a solid polymer to grant the implant a certain rigidity. For example a material such as PEEK is used. PEEK has the advantages of being similar to bone in terms of its mechanical properties, although these parts 2 and 3 can also be manufactured from a metal material such as titanium or stainless steel.

The parts 2 and 3 each comprise means for association, 4 and 5 respectively, with a process. The association means 4 and 5 each comprise a groove, 6 and 7 respectively, and attachment means, 8 and 9 respectively, intended for attachment to the spinous process of the vertebra with which the part is intended to be associated.

In one aspect, the attachment means are intended to surround the spinous process of the vertebra with which the part 2 or 3 is intended to be associated.

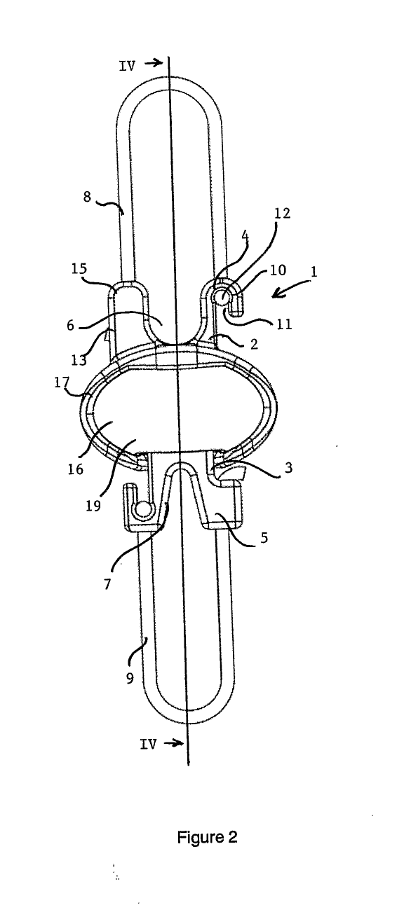

The groove 6 of the top part 2 has a shape that substantially complements the bottom part of the spinous process of the vertebra with which the part 2 is intended to be associated. As shown in FIG. 2, the groove 6 has a substantially U-shaped cross-section. Indeed, the groove 6 is intended to receive the bottom part of a spinous process, the latter being thicker than the top part of this process. The shape of the groove 6 has been adapted to that of the bottom part of the process to avoid major operations on the process when placing the implant. The U-shaped cross-section enables such an adaptation.

The groove 7 of the bottom part 3 has a shape that substantially complements the top part of the spinous process of the vertebra with which the part 3 is intended to be associated. As shown in FIG. 2, the groove 7 has a substantially V-shaped cross-section. Indeed, the groove 7 is intended to accommodate the top part of a spinous process and, as mentioned above, the latter is thinner than the bottom part of this process. The shape of the groove 7 has therefore been adapted to that of the top part of the process, once again, to avoid major operations on the process when placing the implant. The V-shaped cross-section enables such an adaptation.

In a preferred aspect, the attachment means are tightening bands 8, 9. The tightening bands 8 and 9 can be identical for the top 2 and bottom 3 parts, as shown in the figures. This is why only band 8 is described.

The band 8 is associated with the top part 2 on either side of the groove 6 and forms a loop with the groove.

On one side of the groove 6, the band 8 is solidly attached in rotation to the groove by attachment means 10. According to the structure shown in FIGS. 1 and 2, the attachment means 10 comprise a housing 11 arranged on one side of the groove 6 and solidly attached to the latter. The attachment means 10 also comprise a pivot 12 arranged on one end of the tightening band 8 and rotatingly accommodated in the housing 11. Other attachment means 10 are also possible, such as a pivot arranged on one side of the groove 6 passing through a hole made in the end of the band 8 or an external pivot passing through orifices made in one side of the groove 6 and the end of the band 8.

Any attachment means can be used that allow the band 8 to be solidly attached to one side of the groove 6.

On the other side of the groove 6, the band 8 is solidly attached to the groove by tightening means 13 making it possible to adjust the tightness of the band 8 around the spinous process. These tightening means 13 can be of a conventional type, for example with notches 14 on the band 8 cooperating with a projection made in a housing 15 on the other side of the groove 6 as shown in the figures.

In another aspect, the band 8 is solidly attached to the groove by tightening means 13 arranged on either side of the groove.

In one aspect, the tightening bands are made from a polymer and, more particularly, from nylon.

The attachment means 8, 9 are not limited to tightening bands. Indeed, attachment means known to those skilled in the art, such as ligaments, attachment screws or osteoinductive or osteoconductive coatings, can also be used.

It should be noted, however, that tightening bands 8, 9, unlike ligaments, do not stretch and are not abrasive. Thus, tightening bands provide effective support with the spinous process which does not degrade over time.

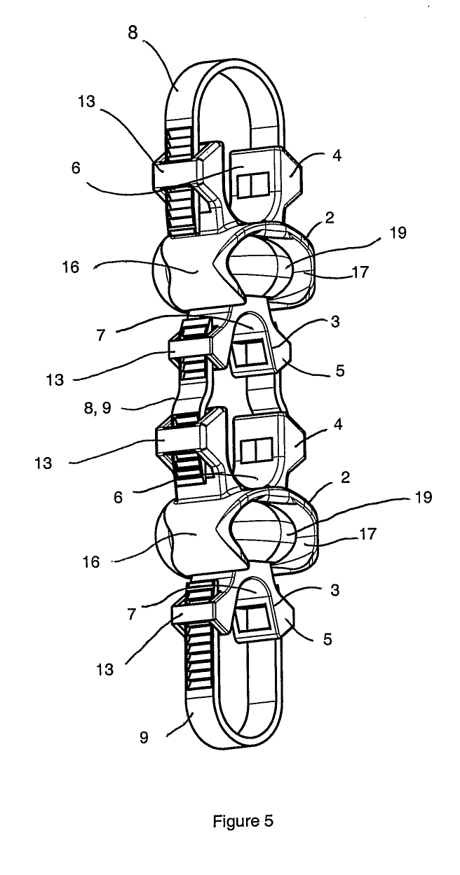

According to one structure shown in FIGS. 5, 6 and 7, the implant 1 can comprise means for association with another implant 1. Thus, it is also possible to make a surgical implant 1 connecting several levels of vertebrae.

As shown in FIG. 5, the means for association between the top part 2 of an implant and the bottom part 3 of the other implant also form means for association with a spinous process. These association means consist, for example, of a tightening band 8 forming association means between the top part 2 of one implant and the bottom part 3 of the other implant. The space created between the two implants can accommodate a spinous process of a vertebra. In this way, three vertebrae are connected by means of surgical implants 1.

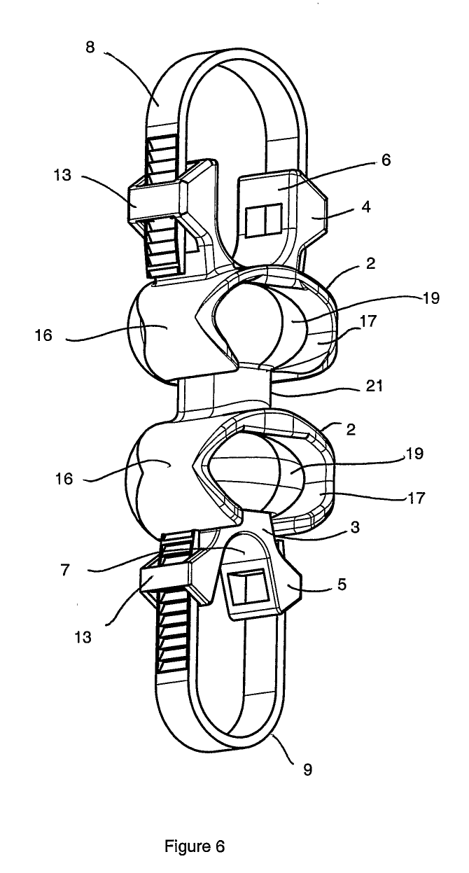

As shown in FIG. 6, the association means between the top part 2 of one implant and the bottom part 3 of another implant, is a connecting rod 21. In fact, in the event of a laminectomy, the means for association between implants do not require a space for accommodating a spinous process.

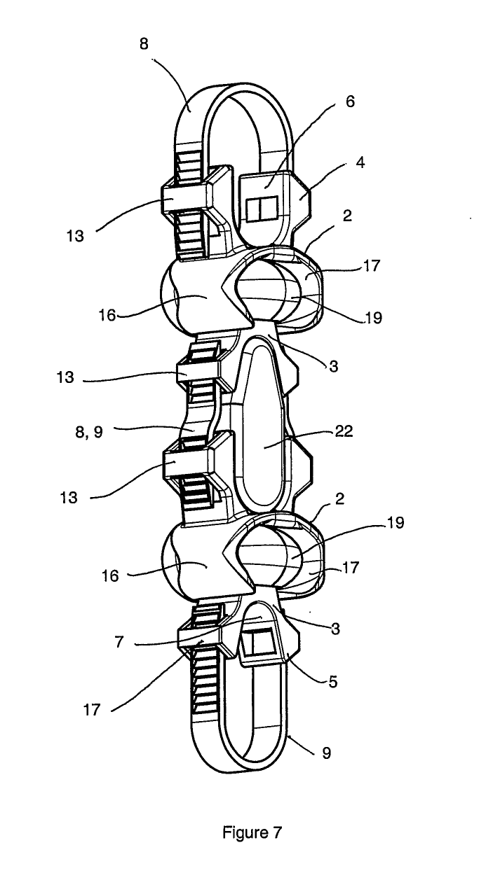

As shown in FIG. 7, the means for association between the top part 2 of one implant and the bottom part 3 of the other implant is a solid intermediate part 22 solidly attached to the top part 2 of one implant and to the bottom part 3 of the other implant by means of one or several attachment means 8, 9.

The parts 2 and 3 comprise, among others, reciprocal means for rotational association 16 with the other part. As shown in the figures, these means 16 form a swivel joint.

For this purpose, the reciprocal association means 16 comprise a housing 17 made on the top part 2 and arranged such as to receive a protuberance 18 of the bottom part 3 in rotation. According to another aspect, not shown, the housing 17 can be made on the bottom part 3 and the protuberance 18 on the top part 2. The association means 16 also comprise a viscoelastic element 19 interposed between the top 2 and bottom 3 parts, arranged such as to absorb the energy from impacts and movements between the vertebrae. The viscoelastic element 19 is, for example, made from a polymer material such as polyurethane (PU) or polycarbonate urethane (PCU).

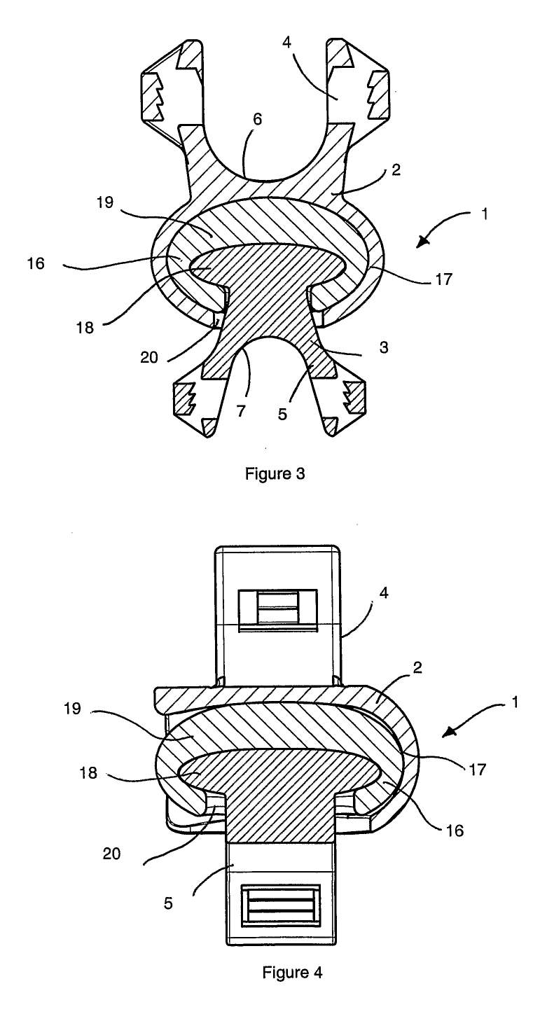

The reciprocal means 16 are arranged so that the rotation only takes place in the direction of movement between the vertebrae. For this reason, the housing 17 has a substantially ovoid cross-section and comprises a slot 20 intended to allow the passage and rotation of the means for association 5 with the spinous process of the bottom part, as shown in FIGS. 3 and 4. On the other hand, the viscoelastic element 19 surrounds the protuberance 18 and has a shape that substantially complements the housing 17, as can be seen in FIGS. 3 and 4. The viscoelastic element 19 is, for example, placed around the protuberance 18. The viscoelastic element 19 “fills” the housing 17, absorbing the energy that may be transmitted from the top part 2 to the bottom part 3 as well as the compression and distraction during an impact between two consecutive vertebrae while allowing relative movement between the vertebrae.

As shown in FIG. 4, the housing 17 is open on one side to allow the assembly of the protuberance 18 and the viscoelastic element 19 inside the housing 17. Both the opening of the housing and the housing 17 itself are shaped so that the protuberance 18 and the viscoelastic element 19 can be inserted in the housing with a slight rotation, with no translational movement. Such an assembly prevents the bottom part 3 from coming loose from the top part 2 by trans-lation once the implant 1 is in place. This therefore guarantees proper support for the parts in relation to one another once the implant has been placed.

The parts 2 and 3 and the viscoelastic element 19 can also be solidly assembled during the manufacturing process, avoiding any untimely separation of the parts.

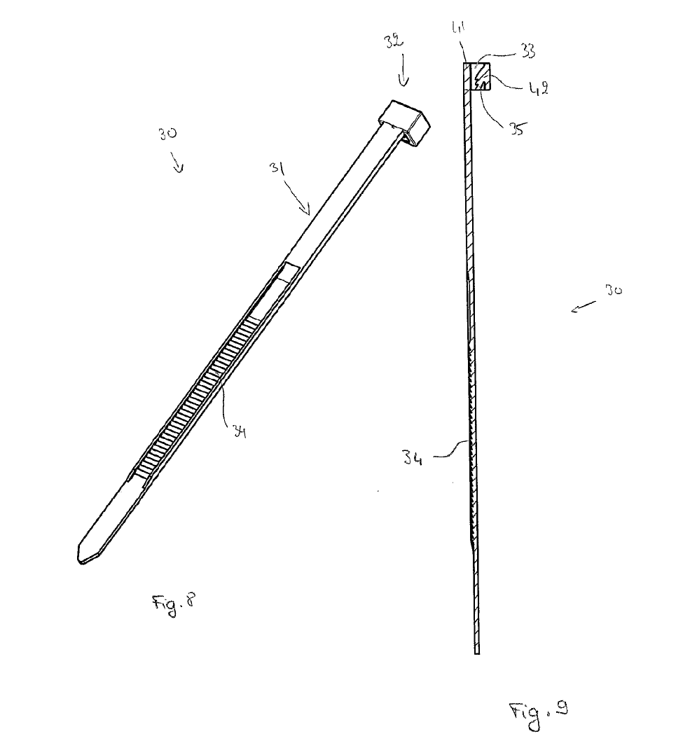

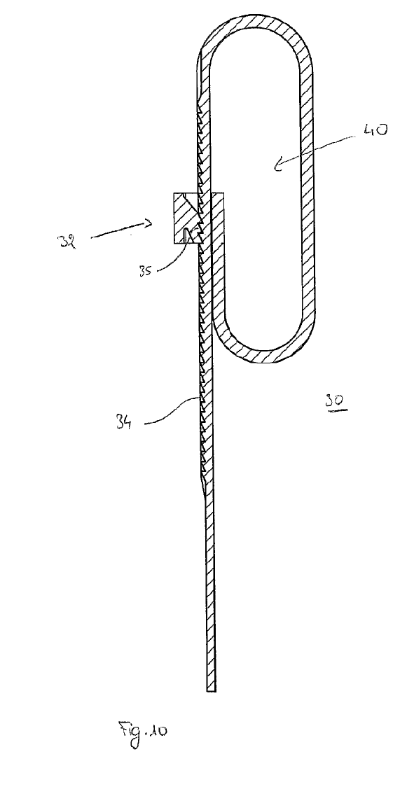

In reference to FIGS. 8 to 14, we describe examples of a system for connecting at least two parts to one another, the parts being either a surgical implant or a bone structure. This therefore involves, with the linking system:

associating implants with one another,

connecting one or more implants to a bone structure, or

connecting bone structures to one another, where the bone structures can possibly be interspersed with one or more implants.

FIGS. 8 to 10 depict a first configuration of such a linking system.

The linking system 30 comprises a flexible strap, preferably in the form of a band 31, at least one end of which is equipped with an attachment element 32 that allows the band 31 to be assembled such as to form a linking loop 40.

For this purpose, the attachment element 32 comprises a hole 33 arranged so that the other end of the band 31 passes through it.

The linking system 30 also comprises tightening means for adjusting the tightness of the band 31 when it is placed around the parts to be connected. These tightening means can be of a conventional type. Thus, they can comprise a notched section 34 on one of the surfaces of the band, notched section which is intended to cooperate with at least one projection 35 made in the hole 33. In the described structure, the hole 33 comprises three consecutive projections.

The hole 33 of the attachment element 32 is delimited by two walls 41, 42, the wall 41 being arranged as an extension of the band 31. The projections 35 are arranged on the inner surface of the wall 42, facing the wall 41.

Likewise, according to one particular aspect, the notched section 34 of the band 31 and the projections 35 of the attachment element 32 are arranged to allow the band 31 to slide in one direction in the attachment element 32 when it passes through the hole 33. The tightening means therefore form irreversible locking means. This locking is made even more reliable by the fact that the attachment element is equipped with several projections.

Advantageously, the notched section 34 is formed on the half of the band 31 that is closest to the free end (end opposite the attachment means 32).

The parts are held together by forming the linking loop 40 around the parts and by tightening it by sliding the band through the hole 33.

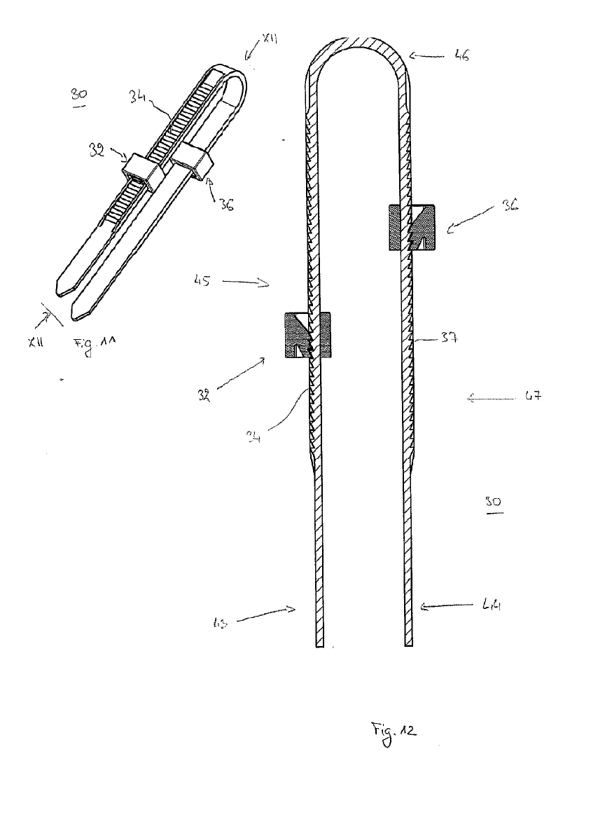

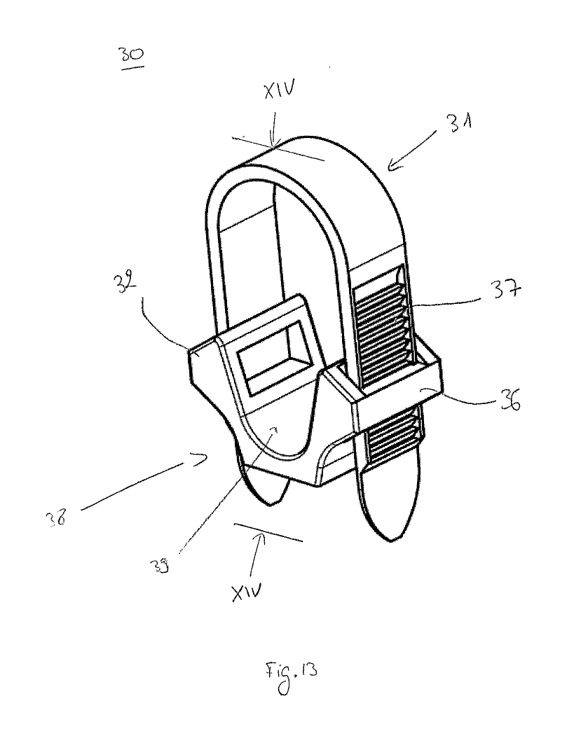

According to a second configuration (see FIGS. 11 and 12), the linking system 30 comprises two attachment elements 32, 36 which one of the ends 43, 44 of the band can respectively pass through. In addition, each attachment element 32, 36 is advantageously configured to slide in one direction on the band, in the direction opposite the end on which it is mounted.

To avoid weakening the section of the band 31 intended to be bent, the band 31 comprises an un-notched section 46. Thus, in the described structure, the band advantageously comprises three sections: two end sections 45, 47 which respectively comprise, on the same side, a partially notched area 34, 37, the end sections 45, 47 being separated by the un-notched section 46, with no notches. Each notched section 34, 37 is intended to cooperate with one of the attachment elements 32, 36.

This second configuration of the attachment system 30 requires at least one of the parts to comprise two splits through which the ends of the band 31 are intended to pass. In the case, for example, of an implant associated with a spinous process of a vertebra, the band 31 is arranged on the process, with the notched surfaces 34 and 37 pointing outwards (in relation to the inside of the linking loop 40). The ends 43 and 44 of the band 31 then each pass through one slot of the implant. The ends 43, 44 then respectively pass through one of the attachment elements 32, 36. The attachment elements 32, 36 are then slid in turns along the band 31 until they come to a stop against the implant, against the opening of the slot from which the band 31 emerges. Each of the strands of the band 31 obtained at the end of the implant, and the respective attachment element, are then pulled tight firmly to hold the implant against the spinous process.

In this configuration, the process and the implant are partially surrounded by the band 31.

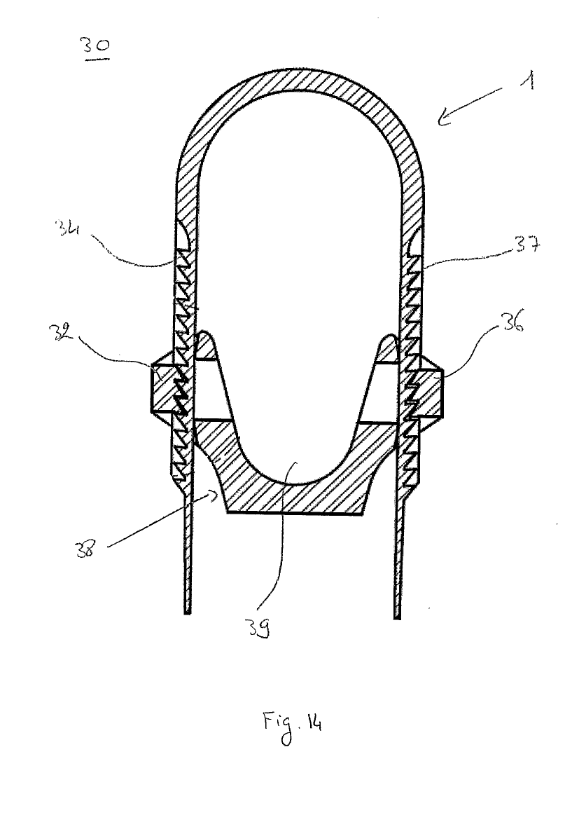

According to a specific configuration (see FIGS. 13 and 14), the attachment elements 32, 36 are connected and arranged such as to form means for association 38 with one of the parts.

The association means 38 advantageously comprise a groove 39 with a shape that substantially complements the section of the part with which they are intended to be associated. In the described embodiment, the groove has a substantially U-shaped cross-section. It is evidently understood that the structure is not limited to such a configuration of the groove and that any other shape with a conformation that complements the part with which it is associated is covered by the scope of the appended claims.

Thus, in the case of association with the bottom part of a spinous process of a vertebra, it would be advantageous to provide association means with a U-shaped groove, the loop surrounding, for example, the top part of the spinous process of one of the top vertebrae or an implant placed between the vertebrae.

In the case of association with the top part of a spinous process of a vertebra, it would be advantageous to provide association means with a V-shaped groove, the loop surrounding, for example, the bottom part of the spinous process of one of the bottom vertebrae or an implant placed between the vertebrae.

The attachment system 30 is advantageously made from a polymer, more particularly from nylon.

1-26. (canceled)

27. A surgical implant that supports and assists movement of adjacent vertebrae comprising: a top part and a bottom part associated with the spinous process of one of the vertebrae, each part comprising means for association with one process and reciprocal means for rotational association with the other part and forming a swivel joint.

28. The surgical implant according to claim 27, further comprising a viscoelastic element interposed between the top and bottom parts.

29. The surgical implant according to claim 28, wherein the reciprocal association means comprise a housing provided on the top or bottom part and arranged to receive a protuberance of the other part in rotation, the viscoelastic element surrounding the protuberance and having a shape that substantially complements the housing.

30. The surgical implant according to claim 29, wherein the housing comprises a slot that allows passage and rotation of the means for association with the spinous process of the other part.

31. The surgical implant according to claim 27, wherein the means for association with the spinous process of the top part comprise a groove with a shape that substantially complements the bottom part of the spinous process of the vertebra with which the part is intended to be associated.

32. The surgical implant according to claim 31, wherein the groove has a substantially U-shaped cross-section.

33. The surgical implant according to claim 27, wherein the means for association with the sinous process of the bottom part comprise a groove with a shape that substantially complements the top part of the spinous process of the vertebra with which the part is intended to be associated.

34. The surgical implant according to claim 33, wherein the groove has a substantially V-shaped cross-section.

35. The surgical implant according to claim 27, wherein the means for association with the spinous process of each part comprise attachment means that attach the spinous process of the vertebra with which the part is intended to be associated.

36. The surgical implant according to claim 35, wherein the attachment means consist of a tightening band.

37. The surgical implant according to claim 36, wherein the tightening band is attached to one side of the groove by tightening means.

38. The surgical implant according to claim 36, wherein the tightening band is attached to both sides of the groove by tightening means.

39. The surgical implant according to claim 28, wherein the viscoelastic element is polyurethane or polycarbonate urethane.

40. The surgical implant according to claim 27, wherein the top and bottom parts are a solid polymer.

41. The surgical implant according to claim 27, further comprising means for association with another implant.

42. The surgical implant according to claim 41, wherein the means for association with another implant also form means for association with a spinous process.

43. A linking system between at least an implant and/or a bone structure comprising: a flexible band, at least one attachment element for the band that attaches the band around all or part of the parts, the attachment element comprising a hole arranged so that the band passes through it, and tightening means that adjust tightness of the band around the parts.

44. The linking system according to claim 43, wherein the tightening means comprise at least one notched section formed on the band that cooperates with at least one projection formed in the hole.

45. The linking system according to claim 44, wherein the notches of the band and the projection of the attachment element are arranged to allow the band to slide in one direction in the attachment element when it passes through the hole.

46. The linking system according to claim 43, wherein the attachment element is made on one end of the band.

47. The linking system according to claim 46, wherein the projection formed in the hole extends in the direction of the surface of the band opposite the notched surface.

48. The linking system according to claim 43, further comprising two attachment elements through which one of the ends of the band can respectively pass, each attachment element configured to slide in one direction on the band, in the direction opposite the end on which it is mounted.

49. The linking system according to claim 48, wherein the band comprises two distinct notched sections on one of its surfaces, each notched section cooperating with one of the attachment elements.

50. The linking system according to claim 48, wherein the attachment elements are connected and arranged with one another to form means for association with one of the parts.

51. The linking system according to claim 50, wherein the association means comprise a groove with a shape that substantially complements a section of the part with which they are intended to be associated.

52. The linking system according to claim 51, wherein the groove has a substantially U-shaped or V-shaped cross-section.
